(19) United States
(12) Patent Application Publication
(10) Pub. No.: US 2006/0250577 A1
(43) Pub. Date: Nov. 9, 2006

(54) Information system for controlling electronic devices with a hierarchically structured operator control menu

(75) Inventors: Stephan Cieler, Frankfurt (DE); Vera Donk, Babenhausen (DE); Guido Meier-Arendt, Langen (DE); Thomas Sulzbach, Offenbach (DE)

Correspondence Address: MORRISON & FOERSTER LLP, 1650 TYSONS BOULEVARD, SUITE 300, MCLEAN, VA, 22102, US

(73) Assignee: Siemens Aktiengesellschaft, Munchen (DE)

(21) Appl. No.: 11/412,070

(22) Filed: Apr. 27, 2006

(30) Foreign Application Priority Data
Apr. 28, 2005 (DE) 10 2005 019 870.8

(51) Int. Cl. A61B 3/14

(52) U.S. Cl. 351/210

(57) ABSTRACT

Information system for controlling electronic devices with a hierarchically structured operator control menu An information system for controlling at least one electronic device with a computing unit which is connected to a memory unit, a control device and a display device, the computing unit reading out, from the memory unit, an operator control menu which is a hierarchically structured operator control menu comprising at least two menu levels, each with at least two menu elements, displaying at least one of the menu levels as the current menu level on the display device, reading in a menu element selected from the current menu level by means of the operator control device, displaying the menu level which is subordinate to the menu element as the menu level which is now current, and graphically indicating the depth of the current menu level within the operator control menu on the display device. In order to improve the clarity and ability to perceive the display, the computing unit causes an outline to be displayed around a graphic display element, the outline representing the depth of the current menu level.

### CLAIM FOR PRIORITY

This application claims the benefit of priority to German Application no. 10 2005 019 870.8, filed in the German language on Apr. 28, 2005, the contents of which are hereby incorporated by reference.

### TECHNICAL FIELD OF THE INVENTION

The invention relates to an information system for controlling at least one electronic device with a computing unit which is connected to a memory unit, a control device and a display device.

### BACKGROUND OF THE INVENTION

DE 103 02 110 A1 discloses a menu-controlled communication device in which a menu content which is divided into menu levels can be displayed on a display device. If it is not possible to display all the menu entries simultaneously on the display device, the possibility of scrolling is provided, i.e. the menu entries are arranged in the form of a list and in each case an excerpt from this list is displayed. The respectively preceding or subsequent invisible element in the list can be moved into the visible excerpt by activating an operator control device. In DE 103 02 110 A1, a running indicator of variable length is used to indicate graphically how many menu entries are contained in the currently displayed menu level. The width of the running indicator is also variable. It indicates which of the menu levels is currently displayed. However, the display of the running indicator reduces the clarity and thus the ability to intuitively perceive the content of the display device.

### SUMMARY OF THE INVENTION

The invention relates to an information system for controlling at least one electronic device with a computing unit which is connected to a memory unit, a control device and a display device, the computing unit reading out, from the memory unit, an operator control menu which is a hierarchically structured operator control menu comprising at least two menu levels, each with at least two menu elements, displaying the menu elements from at least one of the menu levels as a current menu level on the display device, reading in a menu element selected from the current menu level by means of the operator control device, selecting the menu level which is subordinate to the menu element as the menu level which is now current, and displaying its menu elements, and graphically indicating the depth of the current menu level within the operator control menu on the display device.

The present invention therefore specifies an information system of the type mentioned at the beginning with an improved display of the depth of the current menu level.

According to one embodiment of the invention, the computing unit causes an outline to be displayed around a graphic display element, the outline representing the depth of the current menu level. As a result, the addition of a supplementary display element, such as for example the running indicator, is eliminated. The improvement in the clarity which is achieved in this way ensures that the screen content can be perceived more quickly. Since the displayed display elements are retained as they are and only the outline of one or more display elements is varied, a constant graphic appearance is provided, which also improves the ability of the operator to perceive. This is advantageous in particular in environments or in electronic devices in which a user cannot look at the display device continuously. For this reason, the invention is preferably applied in vehicles such as motor vehicles and sailing craft in which a plurality of electronic devices are controlled by means of a common operator control and display device.

In one preferred embodiment of the invention, the outline is formed from a plurality of closed surrounding lines which are located one inside the other, the number of surrounding lines corresponding to the depth of the current menu level. In this context, when there is a change to a deeper menu level, a surrounding line is preferably added, and when there is a change to a higher menu level a surrounding line is removed. In this case, it is possible not only to perceive the menu depth intuitively, as is the case when the width of the running indicator changes, but absolute information is also provided. The structure of the hierarchical operator control menu thus becomes more transparent and is easier for a user to take in.

In a further embodiment, the outline has a different color depending on the depth of the current menu level. The changes in color may be effected here on a single outline or else with respect to the change in the number of surrounding lines. The color coding of the outline increases the ability of the operator to perceive the current menu depth intuitively.

Instead of or together with the change in the color of the outline, in one development it is proposed to produce a spatial effect by means of the outline, as is achieved for example by a shadow display, the level of the graphic display element which is represented by the spatial effect being adapted to the depth of the current menu level. This measure also gives an intuitively more quickly perceived impression of the current menu depth.

In a further embodiment of the invention, the display element which is to be surrounded by the outline represents the operator control device. In this way, the relationship between the change of the current menu level as a result of the activation of the operator control device and the change of the displayed outline is represented graphically and becomes directly comprehensible.

This effect can be enhanced even more if the geometric shape of the graphic display element corresponds to the geometric shape of the operator control device. Thus, for example a circular display element is used in the case of a rotary actuator and a rectangular display element is used in the case of an operator control key.

In one embodiment, the graphic display element which is provided with a variable outline is a box around the menu elements which are assigned to the current menu level. In this way, the information of the menu level is thus linked directly to the display of the menu elements which can be found in this menu level, and this can also be perceived quickly by the user.

In a further embodiment, the size of the graphic display element is adapted to the depth of the current menu level. In the case of the surrounding lines which are located one inside the other, this can be used to keep the external size of the graphic display element, including the surrounding lines, constant by positioning an additional surrounding line directly around the graphic display element and at the same time making the graphic display element smaller. The clarity is thus not adversely affected.

### BRIEF DESCRIPTION OF THE DRAWINGS

The invention will be explained in more detail below with reference to an exemplary embodiments and the drawings, in which:

FIG. 1 shows an information system for controlling two electronic devices in a motor vehicle.

FIG. 2 is an illustration of a first menu level.

FIG. 3 is an illustration of a second menu level.

FIG. 4 is an illustration of a third menu level.

### DETAILED DESCRIPTION OF THE INVENTION

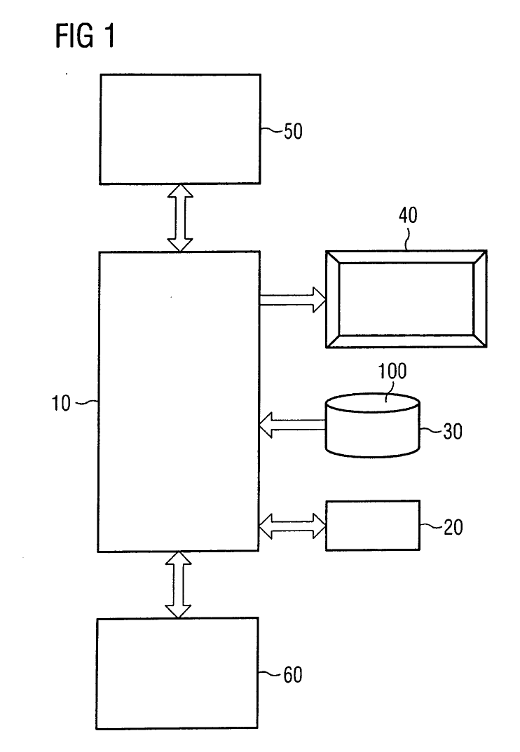

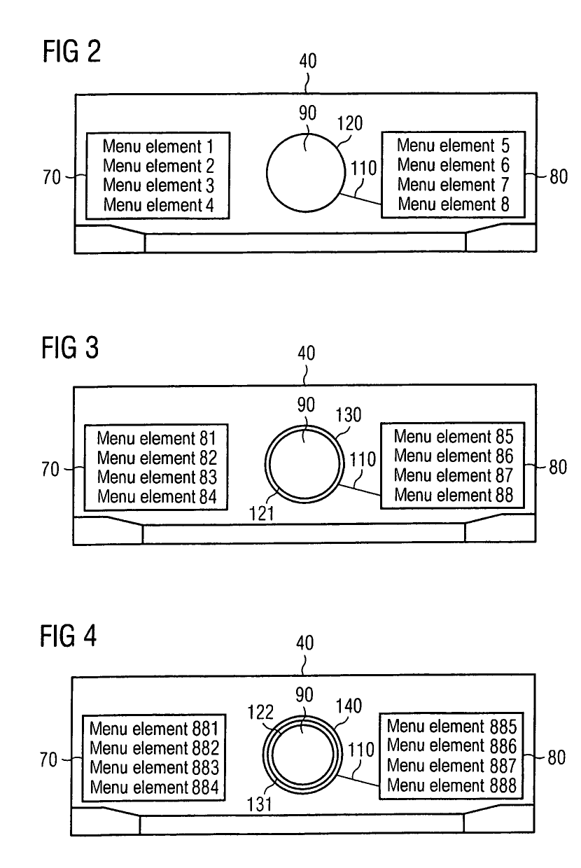

FIG. 1 illustrates an information system including a computing unit 10, a memory unit 20, an operator control device 30 and a display device 40, the information system serving as a first electronic device in a motor vehicle for controlling an air conditioning system 50, and as a second electronic device for controlling an audio system 60. The computing unit 10 is connected to the devices 20, 30, 40 of the information system and to the electronic devices 50, 60. A hierarchically structured operator control menu, which is used to jointly control the air conditioning system 50 and the audio system 60 and contains three menu levels is stored in the memory unit 20. The computing unit 10 causes menu elements of in each case one of the three menu levels to be output on the display device 40, as can be seen in FIGS. 2, 3 and 4. Alternatively, two menu levels can also be displayed simultaneously, but for reasons of clarity this does not occur here. A turn and press actuator is used as the operator control device 30. An operator uses the turn function to select an individual menu element from the displayed menu elements and confirms the selection using the press function.

FIG. 2 illustrates the outputting of the first menu level on the display device 40 in a schematic fashion. The first menu level is understood to be the highest menu level in the hierarchy. Accordingly, the second menu level is the next lowest and the third menu level the lowest menu level. Overall, eight menu levels can be output simultaneously on the display device 40. If more menu elements are included in the current menu level, scrolling is made possible, the scrolling being carried out by means of the turn function of the operator control device 30. The menu elements of the first menu level are characterized by single-digit numbers 1 to 8 in FIG. 2 in order to indicate the menu depth. FIG. 3 shows the menu elements of the second menu level, the menu elements being correspondingly provided with two-digit numbers 81 to 88. And the menu elements of the third menu level in FIG. 4 are indicated by three-digit numbers 881 to 888.

The menu elements of the respective current menu level are distributed between two graphic display elements 70 and 80 which are surrounded by rectangles. Between the graphic display elements 70 and 80 there is a circle 90, as a third graphic display element, with a surrounding line 120, the circle 90 representing the operator control device 30 and having the same geometric shape as the cover surface 100 of the operator control device 30. A pointer element 110 indicates which of the menu elements 1 to 8 is currently selected. In FIG. 2, this is the menu element 8. If the operator confirms the selection of the menu element 8 by pressing the operator control device 30, this information is passed on to the computing unit 10. The computing unit 10 initiates the change into the next lowest menu level, that is to say the second menu level, and causes the menu elements 81 to 88 to be output on the display device 40. At the same time, it adds a further circular surrounding line 130 to the surrounding line 120, the surrounding line 130 being of the same size as the surrounding line 120 from FIG. 2, and the surrounding line 120 being reduced in size into the surrounding line 121. As a result, the size of the graphic display element of the circle 90 has also been correspondingly reduced in size. The operator selects again the last menu element in the current menu level, that is to say the menu element 88 here. After the operator control device 30 has been pressed, the computing unit 10 changes into the third and lowest menu level and displays the menu elements 881 to 888. A further surrounding line 140 is placed around the surrounding lines 121 and 130, and the surrounding lines 130 and 121, and thus also the graphic display element of the circle 90, are reduced in size, producing the surrounding lines 131 and 122. The circle 90 is therefore surrounded by three circular lines located one inside the other in the display of the menu elements of the third menu level.

Instead of or in addition to the multiplication of the surrounding lines around the circle 90, the rectangular boxes around the menu elements, that is to say the graphic display elements 70 and 80, can also be adapted to the depth of the current menu level. In order to indicate the three-level nature of the operator control menu, it is proposed, for example, to display the boxes 70 and 80 in red in the first menu level, in yellow in the second menu level and in green in the third menu level.

1. An information system for controlling at least one electronic device comprising a computing unit which is connected to a memory unit, a control device and a display device, the computing unit
reading out, from the memory unit, an operator control menu which is a hierarchically structured operator control menu comprising at least two menu levels, each with at least two menu elements;
displaying the menu elements from at least one of the menu levels as a current menu level on the display device;
reading in a menu element selected from the current menu level by the operator control device, selecting the menu level which is subordinate to the menu element as the menu level which is now current, and displaying its menu elements; and
graphically indicating the depth of the current menu level within the operator control menu on the display device, wherein the computing unit causes an outline to be displayed around a graphic display element, the outline representing the depth of the current menu level.

2. The arrangement as claimed in claim 1, wherein the outline is formed from a plurality of closed surrounding lines which are located one inside the other, the number of surrounding lines corresponding to the depth of the current menu level.

3. The arrangement as claimed in claim 1, wherein the outline has a different color depending on the depth of the current menu level.

4. The arrangement as claimed in claim 1, wherein a spatial effect is achieved by the outline, the level of the graphic display element which is represented by the spatial effect being adapted to the depth of the current menu level.

5. The arrangement as claimed in claim 1, wherein the graphic display element represents the operator control device.

6. The arrangement as claimed in claim 5, wherein the geometric shape of the graphic display element corresponds to the geometric shape of the operator control device.

7. The arrangement as claimed in claim 1, wherein the graphic display element is a box around the menu elements of the current menu level.

8. The arrangement as claimed in claim 1, wherein the size of the graphic display element is adapted to the depth of the current menu level.

9. A method for controlling at least one electronic device with a computing unit which is connected to a memory unit, a control device and a display device in an information system, the computing unit comprising:
reading out, from the memory unit, an operator control menu which is hierarchically structured operator control menu comprising at least menu levels, each with at least two menu elements;
displaying the menu elements from at least of the menu levels as a current menu level on the display device;
reading in a menu element selected from the current menu level by means of the operator control device, selecting the menu level which is subordinate to the menu element as the menu level which is now current, and displaying its menu elements; and
graphically indicating the depth of the current menu level within the operator control menu on the display device, wherein the computing unit causes an outline to be displayed around a graphic display element, the outline representing the depth of the current menu level.
